(19) United States
(12) Plant Patent Application Publication
(10) Pub. No.: US 2016/0278264 P1
(43) Pub. Date: Sep. 22, 2016

(54) Hop plant named 'HBC 344'

(50) ;

(71) Applicants: Eugene G. Probasco, Yakima, WA (US); Jason Perrault, Toppenish, WA (US)

(72) Inventors: Eugene G. Probasco, Yakima, WA (US); Jason Perrault, Toppenish, WA (US)

(21) Appl. No.: 14/545,738

(22) Filed: Jun. 11, 2015

Related U.S. Application Data
(60) Provisional application No. 62/177,537, filed on Mar. 16, 2015.

(51) Int. Cl. A01H 5/00

(52) U.S. Cl. PLT/236

(57) ABSTRACT

A new hop plant named ‘HBC 344’ is disclosed. The cones of ‘HBC 344’ mature in late September, and yields a crop of 1,600 to 2,000 pounds per acre. ‘HBC 344’ is used for its aromatic quality.

### CROSS-REFERENCE TO RELATED APPLICATIONS

None

### STATEMENT REGARDING FEDERALLY SPONSORED RESEARCH OR DEVELOPMENT

None

### GENUS AND SPECIES

Humulus lupulus

### VARIETY DENOMINATION

‘HBC 344’

### BACKGROUND OF THE INVENTION

‘HBC 344’ is a product of a controlled breeding program carried out by the inventors in the Yakima Valley of Wash. State. ‘HBC 344’ was one of several seedlings resulting from a cross made in 2001 of female parent YCR 5 (U.S. Plant Pat. No. 12,404) and male parent YCR 136M not patented. A single plant of ‘HBC 344’ was selected in 2006, and in 2007 was expanded to 30 plants, which were planted in the area of Toppenish, Wash. The plants were observed and evaluated for several years, and in 2010 were expanded for further observation and evaluation in the Topennish, Wash. area. A 1 acre test plot of ‘HBC 344’ was established in 2011. Throughout several generations of asexual propagation, ‘HBC 344’ has been observed to retain its distinctive characteristics and remain true to type.

### BRIEF DESCRIPTION OF THE PHOTOGRAPH

FIG. 1 illustrates a mature ‘HBC 344’ hop plant grown on a trellis; and

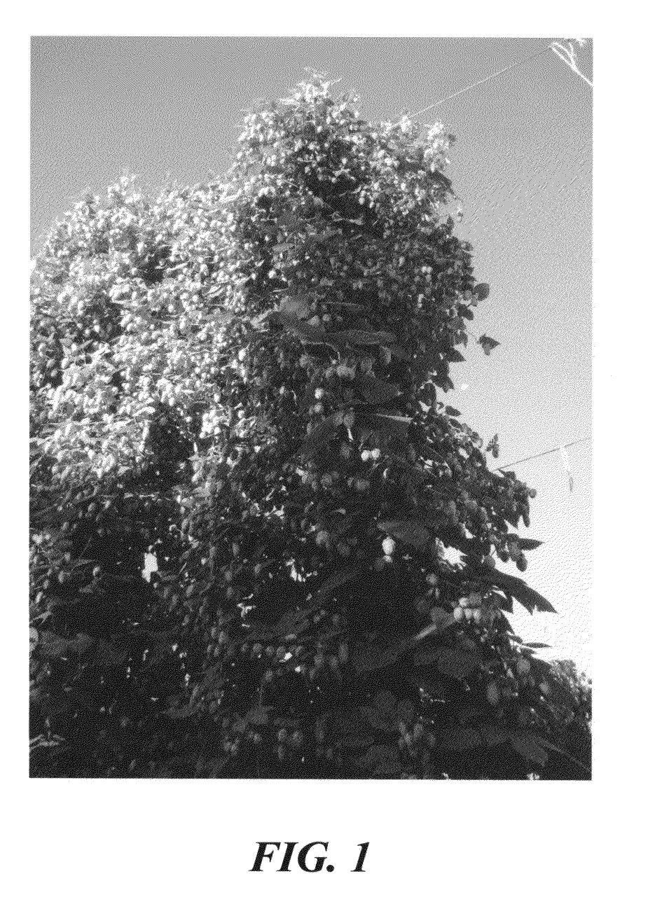

[symbol]

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2011-2014 growing seasons at Toppenish, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. All color references are based on The Royal Horticultural Society Color Chart.
Ploidy: Diploid
Use: Brewing
Harvest date: September 20 to 26 (during 2011 to 2014 growing seasons at Toppenish, Wash.)
Crop yield: 1600 to 2000 pounds per acre
Disease susceptibility: Susceptible to both powdery mildew and downy mildew.
Analytical characteristics: Alpha acid (as % of cone weight) 14.2% to 15.5% Beta acid (as % of cone weight) 4.5% to 5.4% Cohumulone (as % of alpha acids) 24.6% to 29.4%
Bine:
   Color.—144B.
   Stripe present.—Yes.
   Stripe color.—60A.
   Stipule direction.—Up-forked.
   Stipule color.—144D.
   Bine diameter.—8.16 mm at nine feet; 5.33 mm at terminal end of eighteen feet.
Leaf:
   Arrangement.—Opposite.
   Shape.—Palmately Lobed — Cordate.
   Average length of mature leaf.—13.37 cm.
   Average width of mature leaf.—18 cm.
   Color of mature leaf upper surface.—147A.
   Color of mature leaf lower surface.—147B.
   Color of immature leaf upper surface.—147A.
   Color of immature leaf lower surface.—147B.
   Number of lobes.—1-5.
   Margin.—Serrate.
   Serrations per inch.—4.5.
   Average petiole length (mature).—8.87 cm.
   Petiole color at base.—144B, 59A.
   Venation.—Palmate.
   Vein color.—145C.
Cone:
   Avg. length.—3.365 cm.
   Avg. diameter.—1.64 cm.
   Avg. cone weight.—0.77 mg.
   Bract tip color.—145C.
   Bract base color.—149D.
   Bracteole color.—149D.
   Cone shape.—Ovoid.
   Bract shape.—Ovate.
   Bract tip shape.—Cuspidate.
   Bract tip position.—Moderately to tightly appressed.
   Bracteole shape.—Ovate.

1. What is claimed is a new and distinct hop plant as shown and described herein.
